(19) United States
(12) Patent Application Publication
(10) Pub. No.: US 2010/0004240 A1
(43) Pub. Date: Jan. 7, 2010

(54) Indole Compounds

(75) Inventors: Gerard Martin Paul Giblin, Essex (GB); Mairi Gibson, Essex (GB); Adrian Hall, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited, Greenford, Middlesex (GB)

(21) Appl. No.: 12/373,327

(22) PCT Filed: Jul. 9, 2007
(86) PCT No.: PCT/EP07/56946

§ 371 Date: Jan. 12, 2009

(30) Foreign Application Priority Data
Jul. 14, 2006 (GB) 0614066.9

(51) Int. Cl. A61K 31/422; C07D 413/14; A61K 31/5377; A61K 31/4439; A61K 31/437; A61P 25/00

(52) U.S. Cl. 514/235.2; 548/236; 544/137; 546/271.4; 546/118; 546/199; 514/374; 514/339; 514/303; 514/322

(57) ABSTRACT

Compounds of Formula (I) or a pharmaceutically acceptable derivative thereof: wherein R¹, R², and R³ are as defined in the specification, a process for the preparation of such compounds, pharmaceutical compositions comprising such compounds and the use of such compounds in medicine.

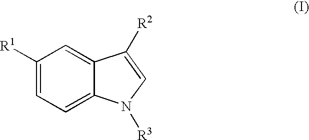

This invention relates to indole compounds, to processes for their preparation, to pharmaceutical compositions containing them and to their use in medicine, in particular their use in the treatment of conditions mediated by the action of PGE₂ at the EP₁ receptor.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion. We have now found a novel group of compounds which bind with high affinity to the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S Narumiya et al, Physiological Reviews 1999, 79(4), 1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. Thus, selective prostaglandin ligands, agonists or antagonists, depending on which prostaglandin E receptor subtype is being considered, have anti-inflammatory, antipyretic and analgesic properties similar to a conventional non-steroidal anti-inflammatory drug, and in addition, inhibit hormone-induced uterine contractions and have anti-cancer effects. These compounds have a diminished ability to induce some of the mechanism-based side effects of NSAIDs which are indiscriminate cyclooxygenase inhibitors. In particular, the compounds have a reduced potential for gastrointestinal toxicity, a reduced potential for renal side effects, a reduced effect on bleeding times and a lessened ability to induce asthma attacks in aspirin-sensitive asthmatic subjects. Moreover, by sparing potentially beneficial prostaglandin pathways, these agents may have enhanced efficacy over NSAIDS and/or COX-2 inhibitors.

In The American Physiological Society (1994, 267, R289-R-294), studies suggest that PGE₂-induced hyperthermia in the rat is mediated predominantly through the EP₁ receptor.

WO 96/06822 (7 Mar. 1996), WO 96/11902 (25 Apr. 1996), EP 752421-A1 (8 Jan. 1997), WO 01/19814 (22 Mar. 2001), WO 03/084917 (16 Oct. 2003), WO 03/101959 (11 Dec. 2003), WO 2004/039753 (13 May 2004), WO 2004/083185 (30 Sep. 2004), WO 2005/037786 (28 Apr. 2005), WO 2005/037793 (28 Apr. 2005), WO 2005/037794 (28 Apr. 2005), WO 2005/040128 (6 May 2005), WO 2005/054191 (16 Jun. 2005), WO2005/108369 (17 Nov. 2005), WO 2006/066968 (29 Jun. 2006), WO 2006/114272 (2 Nov. 2006), WO 2006/114274 (2 Nov. 2006) and WO 2006/114313 (2 Nov. 2006) disclose compounds as being useful in the treatment of prostaglandin mediated diseases.

P. Lacombe et al (220th National Meeting of The American Chemical Society, Washington D.C., USA, 20-24 Aug., 2000) disclosed 2,3-diarylthiophenes as ligands for the human EP₁ prostanoid receptor. Y. Ducharme et al (18^{th} International Symposium on Medicinal Chemistry; Copenhagen, Denmark and Malmo, Sweden; 15^{th}-19^{th} Aug. 2004) disclosed 2,3-diarylthiophenes as EP₁ receptor antagonists. Y. Ducharme et al, Biorg. Med. Chem. Lett., 2005, 15(4): 1155 also discloses 2,3-diarylthiophenes as selective EP₁ receptor antagonists.

S. C. McKeown et al, Bioorg. Med. Chem. Lett., 2007, 17, 1750; A. Hall et al, Bioorg. Med. Chem. Lett., 2007, 17, 1200; A. Hall et al, Bioorg. Med. Chem. Lett., 2007, 17, 916; A. Hall et al, Bioorg. Med. Chem. Lett., 2007, 17, 732; G. M. P. Giblin et al, Bioorg. Med. Chem. Lett., 2007, 17, 385-389; S. C. McKeown et al, Bioorg. Med. Chem. Lett., 2006, 16 (18), 4767-4771; “A. Hall et al, Bioorg. Med. Chem. Lett., 2006, 16 (14), 3657-3662; and A. Hall et al, Bioorg. Med. Chem. Lett., 2006, 16 (10), 2666-2671 relate to EP₁ receptor antagonist compounds.

It is now suggested that a novel group of indole derivatives are indicated to be useful in treating conditions mediated by the action of PGE₂ at EP₁ receptors. Such conditions include pain, or inflammatory, immunological, bone, neurodegenerative or renal disorders.

Accordingly the present invention provides one or more chemical entities selected from compounds of formula (I):

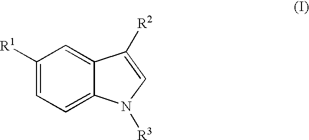

wherein
R¹ represents —CF₃, chlorine or bromine;
R² represents a group of formula (I):

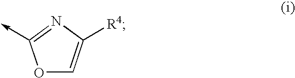

R³ represents isobutyl, —CH₂-cyclopropyl or cyclopentyl;
R⁴ represents —CO—NH—R⁵, —CO-morpholinyl or a group of formula (ii)-(iii):

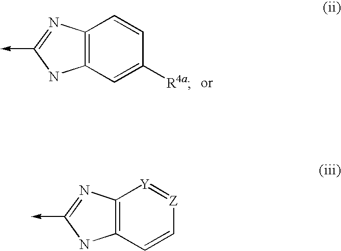

R^{4a} represents hydrogen, —CH₂OH or —CH₂—NR^{a}R^{b};
R⁵ represents hydrogen, pyridyl, morpholinyl, tetrahydropyranyl or —CH₂-tetrahydropyranyl;
R^{a} and R^{b} independently represent hydrogen or C₁₋₃ alkyl or R^{a} and R^{b} together with the nitrogen atom which they are attached form a pyrrolidinyl or piperidinyl ring;
one of Y and Z represents CH and the other represents N;
such that when R² represents a group of formula (I) and R⁴ represents —CO-morpholinyl, R³ represents cyclopentyl;
or derivatives thereof.

Suitably, R¹ represents chlorine.

Compounds of formula (I) include the compounds of Examples 1 to 14 and derivatives thereof.

Particular compounds of the invention include the compounds of Examples 5 and 8 and derivatives thereof.

Certain compounds of the Examples are selective for EP₁ over EP₃. Certain compounds of the Examples have greater than 30 fold selectivity.

Derivatives of the compound of formula (I) include salts, solvates (including hydrates), solvates (including hydrates) of salts, esters and polymorphs of the compound of formula (I). Derivatives of the compounds of formula (I) include pharmaceutically acceptable derivatives.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The present invention also includes isotopically-labelled compounds, which are identical to the compounds of formula (I), except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ²H, ³, ¹¹C, ¹⁴C, ¹⁸F, ³⁵S, ¹²³I and ¹²⁵I.

Compounds of the present invention and pharmaceutically acceptable derivatives (e.g. salts) of said compounds that contain the aforementioned isotopes and/or other isotopes of other atoms are within the scope of the present invention. Isotopically-labelled compounds of the present invention, for example those into which radioactive isotopes such as ³H and/or ¹⁴C are incorporated, are useful in drug and/or substrate tissue distribution assays. ³H and ¹⁴C are considered useful due to their ease of preparation and detectability. ¹¹C and ¹⁸F isotopes are considered useful in PET (positron emission tomography), and ¹²⁵I isotopes are considered useful in SPECT (single photon emission computerized tomography), all useful in brain imaging. Substitution with heavier isotopes such as ²H can afford certain therapeutic advantages resulting from greater metabolic stability, for example increased in vivo half-life or reduced dosage requirements and, hence, are considered useful in some circumstances. Isotopically labelled compounds of formula (I) of this invention can generally be prepared by carrying out the procedures disclosed in the Schemes and/or in the Examples below, by substituting a readily available isotopically labelled reagent for a non-isotopically labelled reagent.

The following definitions are used herein unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I). In one aspect the term “pharmaceutically acceptable derivative” means any pharmaceutically acceptable salt, solvate or solvate of salt. In an alternative aspect the term “pharmaceutically acceptable derivative” means any pharmaceutically acceptable salt.

It will be appreciated that, for pharmaceutical use, the derivatives referred to above will be pharmaceutically acceptable derivatives, but other derivatives may find use, for example in the preparation of compounds of formula (I) and the pharmaceutically acceptable derivatives thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

Compounds of formula (I) can be prepared as set forth in the following schemes and in the examples. The following processes form another aspect of the present invention.

Compounds of formula (I) wherein R⁴ represents —CO—NH—R⁵ may be prepared by the general route shown in Scheme 1 below:

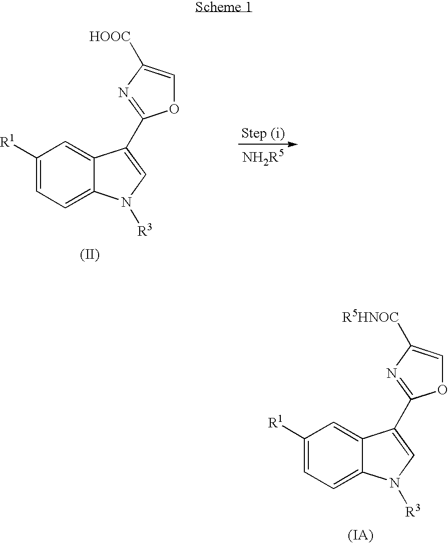

wherein R¹, R³ and R⁵ are as defined above for compounds of formula (I).

Step (i) typically comprises treating a compound of formula (II) with thionyl chloride followed by a compound of formula NH₂—R⁵. Alternatively, step (i) may also be performed in the presence of EDAC, HOBt and a compound of formula NH₂—R⁵.

It will be appreciated that compounds of formula (I) wherein R⁴ represents —CO-morpholinyl may be prepared in an analogous manner to the procedure described in Scheme 1 for compounds wherein R⁴ represents —CO—NH—R⁵.

Compounds of formula (I) wherein R⁴ represents a group of formula (II) and R^{4a} represents hydrogen may be prepared by the general route shown in Scheme 2 below:

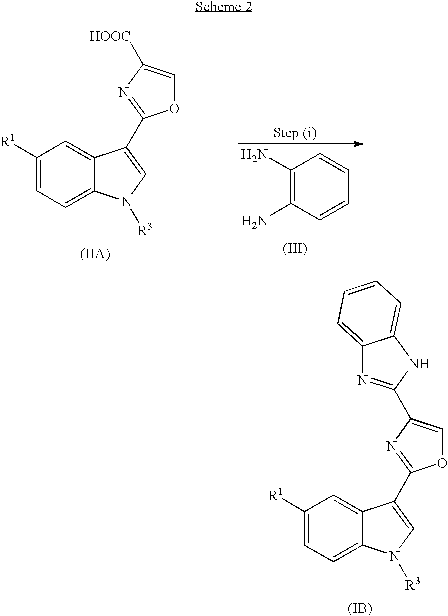

wherein R¹ and R³ are as defined above for compounds of formula (I).

Step (i) typically comprises activation of the carboxylic acid, for example by forming the acid chloride (for example by reaction of the carboxylic acid with phosphorus oxychloride) followed by reaction with a compound of formula (III), followed by dehydration in the presence of a suitable dehydrating reagent e.g. phosphorus oxychloride.

It will be appreciated that compounds of formula (I) wherein R⁴ represents a group of formula (iii) may be prepared in an analogous manner to the procedure described in Scheme 2 for compounds wherein R⁴ represents a group of formula (ii).

Compounds of formula (I) wherein R⁴ represents a group of formula (ii) and R^{4a} represents —CH₂OH or —CH₂—NR^{a}R^{b} may be prepared by the general route shown in Scheme 3 below:

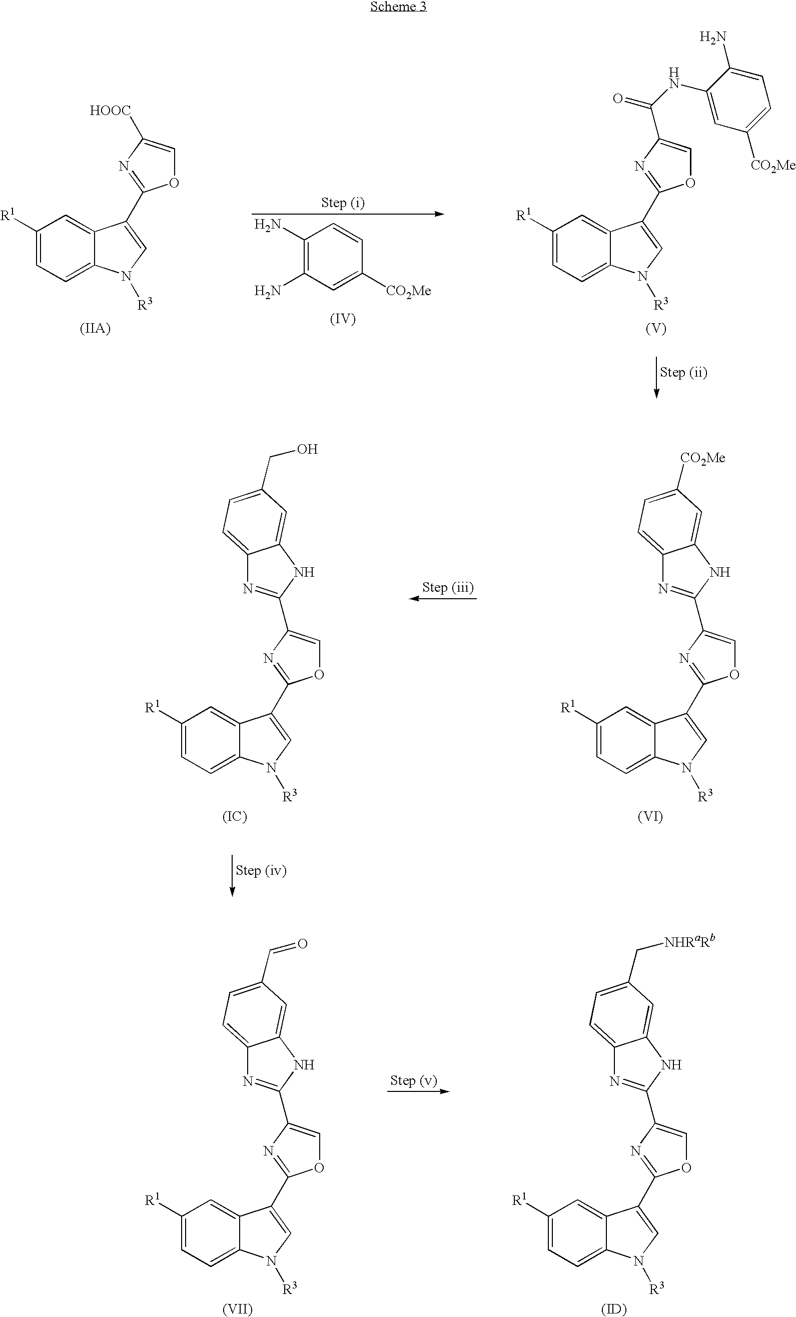

wherein R¹, R³, R^{a} and R^{b} are as defined above.

Step (i) typically comprises reacting a compound of formula (II) with a compound of formula (IV) in the presence of EDAC and HOBt in a suitable solvent e.g. dichloromethane.

Step (ii) typically comprises dehydration of a compound of formula (V), for example by heating in acetic acid.

Step (iii) typically comprises a reduction reaction in the presence of a suitable reducing agent, e.g. lithium aluminium hydride.

Step (iv) typically comprises an oxidation reaction, for example using Dess Martin Period inane.

Step (v) typically comprises reaction of a compound of formula (VII) with a compound of formula NHR^{a}R^{b} in the presence of a suitable reducing agent e.g. sodium triacetoxyborohydride and a suitable acid e.g. acetic acid in a suitable solvent e.g. dichloromethane.

Compounds of formula (IIA) may be prepared the general route shown in Scheme 4 below:

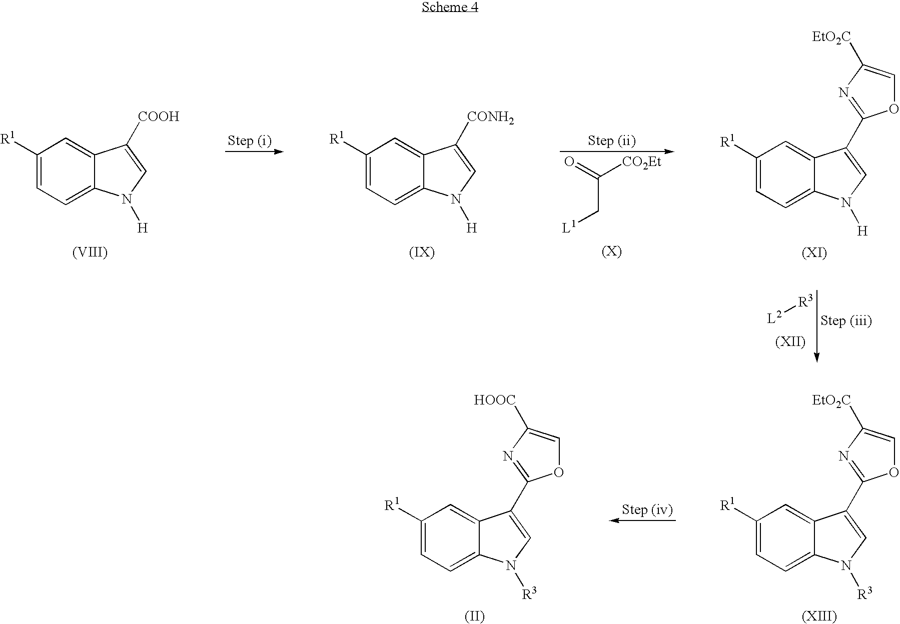

wherein R¹ and R³ are as defined above and L¹ and L² each represent a suitable leaving group, such as a halogen atom (e.g. bromine).

Step (i) typically comprises reacting a compound of formula (VIII) with thionyl chloride followed by ammonia.

Step (ii) typically comprises reacting a compound of formula (IX) with a compound of formula (X) in a suitable solvent e.g. ethanol.

Step (iii) typically comprises reacting a compound of formula (XI) with a compound of formula (XII) in the presence of a base e.g. potassium carbonate, in a suitable solvent e.g. dimethylformamide.

Step (iv) typically comprises treating a compound of formula (XIII) with aqueous sodium hydroxide in an alcoholic solvent, for example methanol or ethanol.

Compounds of formula (IIA) may also be prepared in an analogous manner to Scheme 4 wherein a compound of formula (VII) is reacted with a compound of formula (XI) prior to reaction with a compound of formula (IX).

Certain substituents in any of the reaction intermediates and compounds of formula (I) may be converted to other substituents by conventional methods known to those skilled in the art. Examples of such transformations include the hydrolysis of esters and esterification of carboxylic acids. Such transformations are well known to those skilled in the art and are described in for example, Richard Larock, Comprehensive Organic Transformations, 2nd edition, Wiley-VCH, ISBN 0-471-19031-4.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

Compounds of formula (III), (IV), (VIII), (X) and (XII) are either commercially available, or may be prepared by known methods.

The compounds of the invention bind to the EP₁ receptor and are antagonists of this receptor. They are therefore considered useful in treating conditions mediated by the action of PGE₂ at EP₁ receptors.

One condition mediated by the action of PGE₂ at EP₁ receptors is pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, headache, toothache and dysmenorrhea.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, multiple sclerosis pain, fibromyalgia, HIV-related neuropathy, post-herpetic neuralgia, trigeminal neuralgia, and pain resulting from physical trauma, amputation, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions mediated by the action of PGE₂ at EP₁ receptors include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) and cyclooxygenase-2 (COX-2) inhibitors, cardiovascular diseases; neurodegenerative diseases and neurodegeneration, neurodegeneration following trauma, tinnitus, dependence on a dependence-inducing agent such as opoids (e.g. morphine), CNS depressants (e.g. ethanol), psychostimulants (e.g. cocaine) and nicotine; complications of Type I diabetes, kidney dysfunction, liver dysfunction (e.g. hepatitis, cirrhosis), gastrointestinal dysfunction (e.g. diarrhoea), colon cancer, overactive bladder and urge incontinence.

Inflammatory conditions include skin conditions (e.g. sunburn, burns, eczema, dermatitis, psoriasis), ophthalmic diseases such as glaucoma, retinitis, retinopathies, uveitis and of acute injury to the eye tissue (e.g. conjunctivitis), inflammatory lung disorders (e.g. asthma, bronchitis, emphysema, allergic rhinitis, respiratory distress syndrome, pigeon fancier's disease, farmer's lung, chronic obstructive pulmonary disease (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

Immunological diseases include autoimmune diseases, immunological deficiency diseases or organ transplantation. The compounds of formula (I) are also effective in increasing the latency of HIV infection.

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

Cardiovascular diseases include hypertension or myocardiac ischemia; functional or organic venous insufficiency; varicose therapy; haemorrhoids; and shock states associated with a marked drop in arterial pressure (e.g. septic shock).

Neurodegenerative diseases include dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, particularly mesangial proliferative glomerulonephritis and nephritic syndrome.

The compounds of formula (I) are also considered useful for the preparation of a drug with diuretic action.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof.

A proposed daily dosage of compounds of formula (I) or their pharmaceutically acceptable derivatives for the treatment of man is from 0.01 to 80 mg/kg body weight, more particularly 0.01 to 30 mg/kg body weight per day, for example 0.1 to 10 mg/kg body weight per day, which may be administered as a single or divided dose, for example one to four times per day. The dose range for adult human beings is generally from 8 to 4000 mg/day, more particularly from 8 to 2000 mg/day, such as from 20 to 1000 mg/day, for example 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

The compounds of formula (I) and their pharmaceutically acceptable derivatives may be formulated for administration in any suitable manner. They may be formulated for administration by inhalation or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO99/012930); 5-lipoxygenase inhibitors; NSAIDs (non-steroidal anti-inflammatory drugs) such as diclofenac, indomethacin, nabumetone or ibuprofen; leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists; ligands for the α₂δ-subunit of voltage gated calcium channels, such as gabapentin and pregabalin; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; bradykinin receptor ligands; vanilloid receptor ligand; and purinergic receptor ligands, including antagonists at P2X₃, P2X_{2/3}, P2X₄, P2X₇ or P2X_{4/7}. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

No toxicological effects have currently been observed with the compounds of the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

### Abbreviations

Solid phase extraction (SPE); liquid chromatography/mass spectrometry (LCMS, LC/MS & LC-MS); MDAP (Mass Directed Auto Preparation); NMR (nuclear magnetic resonance); s, d, t, dd, m, b (singlet, doublet, triplet, doublet of doublets, multiplet, broad); Ph, Me, Et, Pr, Bu, Bn (phenyl, methyl, ethyl, propyl, butyl, benzyl), tetrahydrofuran (THF), dichloromethane (DCM), N,N-dimethylformamide (DMF), h (hours), ethylenediaminetetraacetic acid (EDTA), HOBt (1-hydroxybenzotriazole hydrate), 1-(3-diimethylaminopropyl)-3-ethylcarbodiimide hydrochloride (EDC & EDAC), 4-N,N-dimethylaminopyridine (DMAP), dimethylsulfoxide (DMSO), ultraviolet (UV), room temperature (RT, rt), retention time (Rt), minutes (min), EtOAc (ethyl acetate), Et₂O (diethyl ether), MeCN (acetonitrile), EtOH (ethanol), PhCH₃ & PhMe (toluene).

### Purification of Reaction Products

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the Examples may be carried out by conventional methods such as chromatography and/or recrystallisation using suitable solvents. Chromatographic methods are known to the skilled person and include e.g. column chromatography, flash chromatography, HPLC (high performance liquid chromatography), and MDAP (mass directed autopreparation, also referred to as mass directed LCMS purification). MDAP is described in e.g. W. Goetzinger et al, Int. J. Mass Spectrom., 2004, 238, 153-162.

### LCMS

The following LCMS conditions were used during the preparation of the examples.

### Software

Waters MassLynx version 4.0 SP2

### Column

The column used is a Waters Atlantis, the dimensions of which are 4.6 mm×50 mm. The stationary phase particle size is 3 m.

### Solvents

A: Aqueous solvent=Water+0.05% Formic Acid

B: Organic solvent=Acetonitrile+0.05% Formic Acid

### Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

All retention times are measured in minutes.

### Description 1

### 5-Chloro-1H-indole-3-carbonyl chloride (D1)

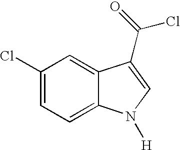

A mixture of 5-chloro-1H-indole-3-carboxylic acid (2.50 g, 12.8 mmol) and thionyl chloride (4.7 ml, 63.9 mmol) was stirred at 60° C. for 30 minutes under an atmosphere of argon. A pink slurry formed. On cooling, further thionyl chloride (1.0 ml, 12.8 mmol) was added and reaction mixture was then reheated to 60° C. and stirred for 1 hour under an atmosphere of argon. After this time, the reaction mixture was allowed to cool and concentrated under reduced pressure to give 5-chloro-1H-indole-3-carbonyl chloride. The residue was used immediately without further purification in the next stage.

### Description 2

### 5-Chloro-1H-indole-3-carboxamide (D2)

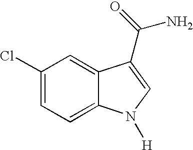

A solution of 5-chloro-1H-indole-3-carbonyl chloride (0.22 g, 1.01 mmol; may be prepared as described in D1) and ammonia in 1,4 dioxane (0.5 M, 8.0 ml, 4.02 mmol) was stirred at room temperature for 30 minutes under an atmosphere of argon. After this time, the solvent was evaporated under reduced pressure. The residue was partitioned between EtOAc and NaHCO₃ (sat. aq solution). The organics were dried over magnesium sulfate, filtered and concentrated under reduced pressure to give a white solid, 5-chloro-1H-indole-3-carboxamide (0.18 g, 92%). Residue used without further purification in the next stage. LCMS Rt=1.92 min, [MH⁺] 195

### Description 3

### Ethyl 2-(5-chloro-1H-indol-3-yl)-1,3-oxazol-4-carboxylate (D3)

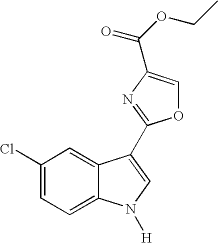

A solution of 5-chloro-1H-indole-3-carboxamide (1.03 g, 5.30 mmol; may be prepared as described in D2) and ethyl 3-bromo-2-oxopropanoate (0.66 ml, 5.30 mmol) in EtOH (11 ml) was stirred at 80° C. for a maximum time of 16 hours (overnight) under an atmosphere of argon. The reaction was monitored by LC-MS. After this time, the reaction mixture was concentrated under reduced pressure. The residue was diluted with EtOAc and washed with water (×2). The organics were dried over magnesium sulfate, filtered and concentrated under reduced pressure to give a brown solid. The residue was recrystallised from EtOAc and Hexane to give ethyl 2-(5-chloro-1H-indol-3-yl)-1,3-oxazole-4-carboxylate (0.46 g, 30%).

LCMS Rt=3.00 min, [M+H] 291, 293.

### Description 4

### Ethyl 2-[5-chloro-1-(cyclopropylmethyl)-1H-indol-3-yl]-1,3-oxazole-4-carboxylate (D4)

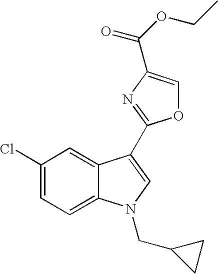

A mixture of ethyl 2-(5-chloro-1H-indol-3-yl)-1,3-oxazole-4-carboxylate (0.060 g, 0.207 mmol; may be prepared as described in D3) and potassium carbonate (0.057 g, 0.414 mmol) in dry DMF (0.5 ml) was stirred at room temperature. (Bromomethyl)cyclopropane (80 μl, 0.828 mmol) was added and the reaction mixture was stirred at room temperature for 17 hours (overnight) under an atmosphere of argon. The reaction was monitored by LC-MS. After this time, the reaction mixture was diluted with EtOAc and washed with water. The organics were dried over magnesium sulfate, filtered and concentrated under reduced pressure to give colourless oil. Where purification was required, column chromatography [SiO₂, Hexane:EtOAc (9:1 to 2:3)] to give ethyl 2-[5-chloro-1-(cyclopropylmethyl)-1H-indol-3-yl]-1,3-oxazole-4-carboxylate. (58 mg, 82%)

LCMS Rt=3.61 min, [M+H]: 345, 347

In some cases, further alkyl bromide (4 equiv) was added and heating to 50° C. for a maximum of 17 hours (overnight) was required to progress the reaction further to completion.

### Description 5

### Ethyl 2-(5-chloro-1-cyclopentyl-1H-indol-3-yl)-1,3-oxazole-4-carboxylate (D5)

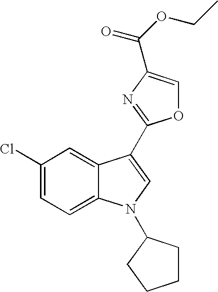

D5 was prepared using an analogous procedure to that described in D4.

LCMS Rt=3.90 min, [MH⁺] 359, 361

### Description 6

### 2-[5-Chloro-1-(cyclopropylmethyl)-1H-indol-3-yl]-1,3-oxazole-4-carboxylic acid (D6)

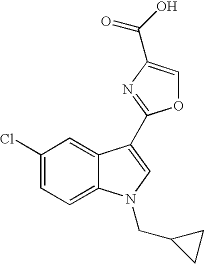

A solution of ethyl 2-[5-chloro-1-(cyclopropylmethyl)-1H-indol-3-yl]-1,3-oxazole-4-carboxylate (may be prepared as described in D4) and 2M sodium hydroxide (4.0 eq.) in EtOH (2.7 ml) was stirred at 90° C. for 30 minutes to 18 hours (overnight). The reaction was monitored by LC-MS. After this time, the reaction mixture was allowed to cool to room temperature and concentrated under reduced pressure. The residue was diluted with water and acidified to pH 1 using 2M HCl. The product was extracted with EtOAc. The combined organics were dried over magnesium sulfate, filtered and concentrated under reduced pressure. Where purification was required, Mass Directed Automated Purification (MDAP) was used. The resulting residue was triturated using hexane to give a white solid, 2-[5-chloro-1-(cyclopropylmethyl)-1H-indol-3-yl]-1,3-oxazole-4-carboxylic acid (26 mg, 48

LCMS Rt=3.01 min, [M+H] 317, 319.

### Description 7

### 2-(5-Chloro-1-cyclopentyl-1H-indol-3-yl)-1,3-oxazole-4-carboxylic acid (D7)

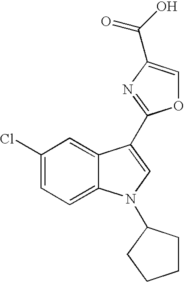

D7 was prepared from D5 using an analogous procedure to that described in D6.

LCMS Rt=3.20 min [MH⁺]: 331, 333.

### Description 8

### 2-Methylpropyl 5-chloro-1-(2-methylpropyl)-1H-indole-3-carboxylate (D8)

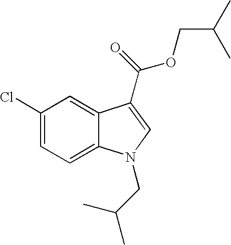

A solution of 5-chloroindole-3-carboxylic acid (2.00 g, 10.26 mmol) in dry DMF (35 ml) was stirred at room temperature under an atmosphere of argon. K₂CO₃ (2.831 g, 20.52 mmol) was added. 1-bromo-2-methyl propane (4.462 ml, 41.04 mmol) was added to the solution, and the solution heated to 60° C. overnight. The mixture was cooled to room temperature and then diluted with EtOAc (˜200 ml). The organics were washed with H₂O (×3), dried over MgSO₄, filtered and concentrated under reduced pressure to give a pink coloured solid. The residue was chromatographed [SiO₂, Hexane:EtOAc 0-25%] to give product (2.217 g, 70% yield).

LCMS Rt=4.03 min, [MH⁺] 308, 310.

### Description 9

### 5-Chloro-1-(2-methylpropyl)-1H-indole-3-carboxylic acid (D9)

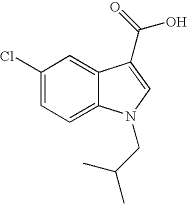

A solution of 2-methylpropyl 5-chloro-1-(2-methylpropyl)-1H-indole-3-carboxylate (2.217 g, 7.22 mmol; may be prepared as described in D8) in EtOH (10.0 ml) was stirred at room temperature. NaOH (2M, 10.0 ml) was added, at this stage a precipitate formed. The mixture was heated to 60° C. for ˜16 hours (overnight). After this time, the solution was cooled to room temperature and the solvent was removed under reduced pressure. The solution was acidified using 2M HCl and organics extracted into EtOAc. Combined organics were washed with water (×2), dried over MgSO₄, filtered and concentrated under reduced pressure to give a white solid (1.711 g, 95% yield)

LCMS Rt=2.96 min, [MH⁺] 252, 254.

### Description 10

### 5-Chloro-1-(2-methylpropyl)-1H-indole-3-carbonyl chloride (D10)

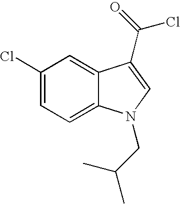

A solution of 5-chloro-1-(2-methylpropyl)-1H-indole-3-carboxylic acid (1.711 g, 6.82 mmol; may be prepared as described in D9) in SOCl₂ (3.0 ml) was heated to 60° C. for 1½ hours. After this time, solution was cooled to room temperature the mixture was concentrated under reduced pressure. The residue was used directly, without purification.

### Description 11

### 5-Chloro-1-(2-methylpropyl)-1H-indole-3-carboxamide (D11)

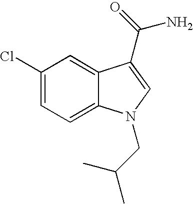

A solution of 5-chloro-1-(2-methylpropyl)-1H-indole-3-carbonyl chloride (6.82 mmol; may be prepared as described in D10) in NH₄OH (4.0 ml) was stirred at 0° C. for 5 minutes. The solvent was removed under reduced pressure to give a light brown coloured solid. The residue was used without further purification.

LCMS Rt=2.68 min, [MH⁺] 251, 253.

### Description 12

### Ethyl 2-[5-chloro-1-(2-methylpropyl)-1H-indol-3-yl]-1,3-oxazole-4-carboxylate (D12)

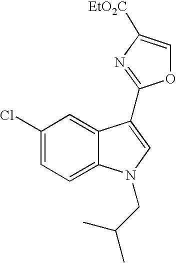

A solution of 5-chloro-1-(2-methylpropyl)-1H-indole-3-carboxamide (1.705 g, 6.82 mmol; may be prepared as described in D11) in EtOH (15.0 ml) was stirred at 60° C. for ˜16 hours (overnight) under an atmosphere of argon. The solution was cooled to room temperature and the solvent removed under reduced pressure. The residue was partitioned between EtOAc (˜200 ml) and H₂O (˜100 ml). Organics were washed with water (2×100 ml), dried over MgSO₄, filtered and concentrated under reduced pressure to give a brown oil. The residue was chromatographed [SiO₂ Hexane:EtOAc 3:1] to give product (2.161 g, 92% yield).

LCMS Rt=3.77 min, [MH⁺] 347, 349.

### Description 13

### 2-[5-Chloro-1-(2-methylpropyl)-1H-indol-3-yl]-1,3-oxazole-4-carboxylic acid (D13)

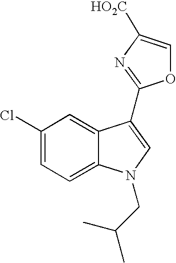

A solution of ethyl 2-[5-chloro-1-(2-methylpropyl)-1H-indol-3-yl]-1,3-oxazole-4-carboxylate (2.161 g, 6.82 mmol; may be prepared as described in D12) in EtOH (10.0 ml) was stirred at room temperature. NaOH (2M, 5.0 ml) was added and the solution heated to 60° C. overnight. The solution was cooled to room temperature and the solvent removed under reduced pressure. The mixture was acidified using 2M HCl, and the organics were extracted into EtOAc. The combined organics were washed with H₂O (×2), dried over MgSO₄, filtered and concentrated under reduced pressure to give a yellow coloured oil. LCMS Rt=3.12 min, [MH⁺] 319, 321.

### Description 14

### 2-[5-Chloro-1-(2-methylpropyl)-1H-indol-3-yl]-1,3-oxazole-4-carbonyl chloride (D14)

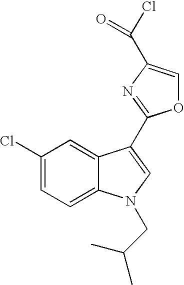

A solution of 2-[5-chloro-1-(2-methylpropyl)-1H-indol-3-yl]-1,3-oxazole-4-carboxylic acid (0.213 g, 0.67 mmol; may be prepared as described in D13) was stirred at room temperature under an atmosphere of argon. SOCl₂ (1.5 ml) was added and the solution was stirred at room temperature for 1½ hours. Solution was then heated to 60° C. for 1 hour. After this time the solvent was removed under reduced pressure. The residue was used directly without purification.

### Description 15

### Methyl 4-amino-3-[({2-[5-chloro-1-(2-methylpropyl)-1H-indol-3-yl]-1,3-oxazol-4-yl}carbonyl)amino]benzoate (D15)

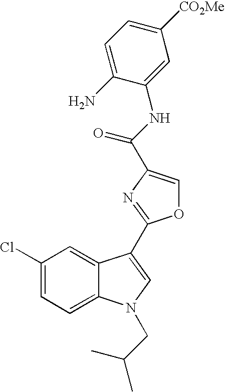

A solution of 2-[5-chloro-1-(2-methylpropyl)-1H-indol-3-yl]-1,3-oxazole-4-carboxylic acid (0.300 g, 0.94 mmol; may be prepared as described in D13) in DCM (3.1 ml) was stirred at room temperature under an atmosphere of argon. EDAC (0.215 g, 1.13 mmol) was added and the solution stirred for 5 minutes. HOBt (0.173 g, 1.13 mmol) was added and the solution stirred for a further 10 minutes. Methyl-3,4-diamine benzoate (0.188 g, 1.13 mmol) was added and the solution stirred at room temperature overnight. The solution was diluted with DCM (˜30 ml) and washed with water (3×20 ml). The organics were dried over MgSO₄, filtered and concentrated under reduced pressure to give a brown oil. The residue was chromatographed [SiO₂, Hexane:EtOAc 36-56%] to give pure product (0.232 g).

LCMS Rt=3.48 min, [MH⁺] 467, 469.

### Description 16

### Methyl 2-{2-[5-chloro-1-(2-methylpropyl)-1H-indol-3-yl]-1,3-oxazol-4-yl}-1H-benzimidazole-5-carboxylate (D16)

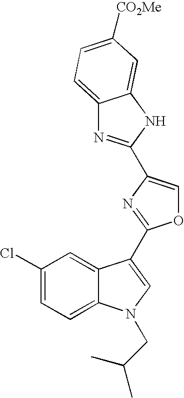

A solution of methyl 4-amino-3-[({2-[5-chloro-1-(2-methylpropyl)-1H-indol-3-yl]-1,3-oxazol-4-yl}carbonyl)amino]benzoate (0.232 g, 0.5 mmol; may be prepared as described in D15) in AcOH (4.0 ml) was stirred at 120° C. under an atmosphere of argon for 2Y2 hours. The solution was cooled to room temperature. Solution was diluted with EtOAc (˜50 ml) and washed with water (3×50 ml). Organics were dried over MgSO₄, filtered and concentrated under reduced pressure to give a yellow oil.

LCMS Rt=3.70 min, [MH⁺] 449, 451.

### Description 17

### 2-{2-[5-Chloro-1-(2-methylpropyl)-1H-indol-3-yl]-1,3-oxazol-4-yl}-1H-benzimidazole-5-carbaldehyde (D17)

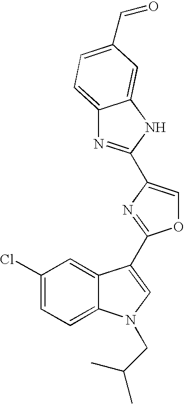

A solution of (2-{2-[5-chloro-1-(2-methylpropyl)-1H-indol-3-yl]-1,3-oxazol-4-yl}-1H-benzimidazol-5-yl)methanol (0.200 g, 0.47 mmol; may be prepared as described in E11) in dry DCM (2.4 ml) was stirred at 0° C. under an atmosphere of argon. Dess-Martin Periodinane (0.202 g, 0.47 mmol) was added to the cooled solution. Mixture was stirred at 0° C. for 3¼ hours. Na₂S₂O₃ (10% aq. soln., 11 ml) and NaHCO₃ (sat. aq. soln., 11 ml) were added to the mixture at 0° C. The mixture was allowed to warm to room temperature. The organics were extracted into DCM (×2). The combined organics were dried over MgSO₄, filtered and concentrated under reduced pressure to give a brown coloured oil. LCMS Rt=3.50 min, [MH⁺] 419, 421.

### Example 1

### 2-[5-Chloro-1-(cyclopropylmethyl)-1H-indol-3-yl]-N-(tetrahydro-2H-pyran-4-yl)-1,3-oxazole-4-carboxamide (E1)

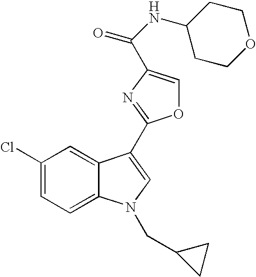

A solution of 2-[5-chloro-1-(cyclopropylmethyl)-1H-indol-3-yl]-1,3-oxazole-4-carboxylic acid (0.050 g, 0.158 mmol; may be prepared as described in D6) in DCM (0.6 ml) was stirred at room temperature under an atmosphere of argon. N-ethylcarbodiimide hydrochloride (EDAC) (0.036 g, 0.190 mmol), 1-hydroxybenzotriazole (HOBt) (0.026 g, 0.190 mmol) and tetrahydro-2H-pyran-4-ylamine (0.020 mg, 0.190 mmol) were added to the stirred solution. Reaction mixture was stirred at room temperature under an atmosphere of argon for 1 hour. The reaction was monitored by LC-MS. Reaction mixture left at room temperature under an atmosphere of argon for a further 17 hours (overnight). After this time, further tetrahydro-2H-pyran-4-ylamine (1.2 eq., 0.020 mg, 0.190 mmol) was added to the reaction mixture. The reaction mixture was left stirring at room temperature for further 3 hours. After this time, the reaction mixture was diluted with excess DCM and washed with NaHCO₃ (sat. aq solution). Organic layer was washed with water (3×10 ml). Organics were dried over magnesium sulfate, filtered and concentrated under reduced pressure. Where purification was required, Mass Directed Automated Purification (MDAP) was used. The resulting residue was triturated using hexane to give a white solid, 2-[5-chloro-1-(cyclopropylmethyl)-1H-indol-3-yl]-N-(tetrahydro-2H-pyran-4-yl)-1,3-oxazole-4-carboxamide (10 mg, 16%).

LCMS Rt=3.20 min, [MH⁺] 400, 402.

### Example 2

### 2-[5-Chloro-1-(cyclopropylmethyl)-1H-indol-3-yl]-N-4-morpholinyl-1,3-oxazole-4-carboxamide (E2)

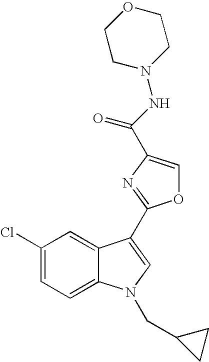

E2 was prepared using an analogous procedure to that described in E1.

LCMS Rt=2.95 min, [MH⁺] 400, 402.

### Examples 34

### 2-(5-Chloro-1-cyclopentyl-1H-indol-3-yl)-N-4-morpholinyl-1,3-oxazole-4-carboxamide and 5-Chloro-1-cyclopentyl-3-[4-(4-morpholinylcarbonyl)-1,3-oxazol-2-yl]-1H-indole (E3-E4)

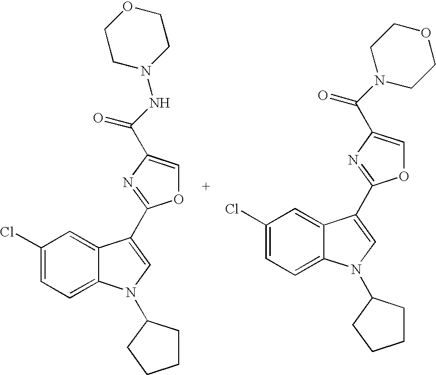

A solution of 2-(5-chloro-1-cyclopentyl-1H-indol-3-yl)-1,3-oxazole-4-carboxylic acid (0.060 g, 0.182 mmol; may be prepared as described in D7) in DCM (0.6 ml) was stirred at room temperature under an atmosphere of argon. N-ethylcarbodiimide hydrochloride (EDAC) (0.042 g, 0.218 mmol), 1-hydroxybenzotriazole (HOBt) (0.030 g, 0.218 mmol) and 4-morpholinamine (0.021 ml, 0.218 mmol) were added to the stirred solution. Reaction mixture was stirred at room temperature under an atmosphere of argon for 1 hour. The reaction was monitored by LC-MS. Reaction mixture left at room temperature under an atmosphere of argon for a further 17 hours (overnight). After this time, further 4-morpholinamine (1.2 eq, 0.021 ml, 0.218 mmol) was added to the reaction mixture. The reaction mixture was left stirring at room temperature for further 3 hours. After this time, the reaction mixture was diluted with excess DCM and washed with NaHCO₃ (sat. aq solution). Organic layer was washed with water (3×10 ml). Organics were dried over magnesium sulfate, filtered and concentrated under reduced pressure. The two products were separated using Mass Directed Automated Purification, MDAP. The resulting residues were triturated using hexane to give two white solids, 2-(5-chloro-1-cyclopentyl-1H-indol-3-yl)-N-4-morpholinyl-1,3-oxazole-4-carboxamide (11 mg, 15%) and 5-chloro-1-cyclopentyl-3-[4-(4-morpholinylcarbonyl)-1,3-oxazol-2-yl]-1H-indole (7 mg)

LCMS Rt=3.17 min, [MH⁺] 414, 416 and LCMS Rt=3.42 min, [MH⁺] 400, 402 respectively.

### Example 5

### 2-[5-Chloro-1-(2-methylpropyl)-1H-indol-3-yl]-N-3-pyridinyl-1,3-oxazole-4-carboxamide (E5)

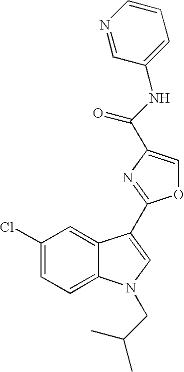

A solution of 2-[5-chloro-1-(2-methylpropyl)-1H-indol-3-yl]-1,3-oxazole-4-carbonyl chloride (0.038 g, 0.11 mmol; may be prepared as described in D14) in dry DCM (0.5 ml) was stirred at room temperature under an atmosphere of argon. Et₃N (0.018 ml, 0.13 mmol) was added, then 3-amino pyridine (0.01 g, 0.13 mmol) was added. The solution was stirred at room temperature for 1 hour. After this time, water was added to the solution and the organics separated using a phase separator cartridge. The organics were concentrated under reduced pressure. The crude product was purified using MDAP to give pure product (8 mg).

LCMS Rt=3.14 min, [MH⁺] 395, 397.

### Examples 6-8 (E6-E8)

The following compounds were prepared using an analogous procedure to that described in E5:

| Name | Structure | Data |
|---|---|---|
| 2-[5-Chloro-1-(2- methylpropyl)-1H-indol-3-yl]- N-2-pyridinyl-1,3-oxazole-4- carboxamide (E6) | 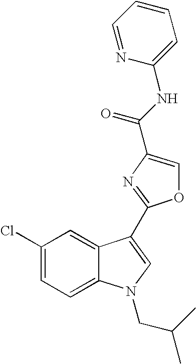 | LCMS Rt = 3.88 min, [MH⁺] 395, 397 |
| 2-[5-Chloro-1-(2- methylpropyl)-1H-indol-3-yl]- N-(tetrahydro-2H-pyran-4- ylmethyl)-1,3-oxazole-4- carboxamide (E7) | 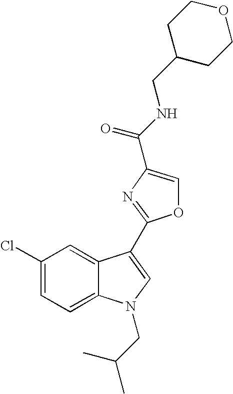 | LCMS Rt = 3.42 min, [MH⁺] 416, 418 |
| 2-[5-Chloro-1-(2- methylpropyl)-1H-indol-3- yl]-1,3-oxazole-4- carboxamide (E8) | 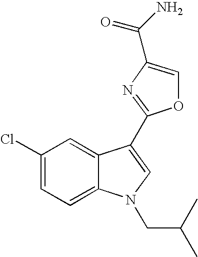 | LCMS Rt = 3.11 min, [MH⁺] 319, 320 |

### Example 9

### 2-{2-[5-Chloro-1-(2-methylpropyl)-1H-indol-3-yl]-1,3-oxazol-4-yl}-1H-benzimidazole hydrochloride (E9)

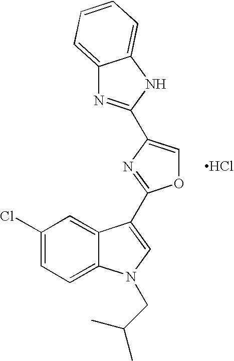

A solution of 2-[5-chloro-1-(2-methylpropyl)-1H-indol-3-yl]-1,3-oxazole-4-carboxylic acid (0.050 g, 0.16 mmol; may be prepared as described in D13) in POCl₃ (1.0 ml) was stirred at room temperature under an atmosphere of argon. 1,2-phenylenediamine (0.017 g, 0.16 mmol) was added to the solution and the solution heated to 120° C. for ˜63 hours (over weekend). POCl₃ was quenched by addition of the mixture to 5M NaOH. The organics were extracted into EtOAc and washed with water. The organics were dried over MgSO₄, filtered and concentrated under reduced pressure. Samples were purified using MDAP, and then treated with HCl in Et₂O to give product (0.017 g).

LCMS Rt=3.13 min, [MH⁺] 391, 393.

### Example 10

### 2-{2-[5-Chloro-1-(2-methylpropyl)-1H-indol-3-yl]-1,3-oxazol-4-yl}-1H-imidazo[4,5-b]pyridine (E10)

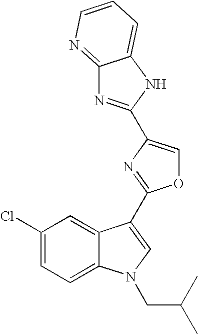

E10 was prepared using an analogous procedure to that described in E9.

LCMS Rt=2.38 min, [MH⁺] 392, 394.

### Example 11

### (2-{2-[5-Chloro-1-(2-methylpropyl)-1H-indol-3-yl]-1,3-oxazol-4-yl}-1H-benzimidazol-5-yl)methanol (E11)

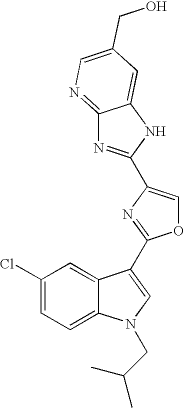

A solution of methyl 2-{2-[5-chloro-1-(2-methylpropyl)-1H-indol-3-yl]-1,3-oxazol-4-yl}-1H-benzimidazole-5-carboxylate (0.200 g, 0.45 mmol; may be prepared as described in D16) in THF (2.5 ml) was added to a stirred solution of LiAlH₄ (1M in THF, 0.27 ml, 0.27 mmol) in THF (2.0 ml) at 0° C. under an atmosphere of argon overnight. More LiAlH₄ (1M in THF, 0.27 ml, 0.27 mmol) was added and the mixture stirred for a further 3 hours at room temperature. H₂O (few drops) was added to quench the mixture. Organics were extracted into EtOAc and washed with water. Organics were dried over MgSO₄, filtered and concentrated under reduced pressure to give a yellow oil.

LCMS Rt=2.73 min, [MH⁺] 421, 423.

### Example 12

### 2-{2-[5-Chloro-1-(2-methylpropyl)-1H-indol-3-yl]-1,3-oxazol-4-yl}-5-(1-piperidinylmethyl)-1H-benzimidazole dihydrochloride (E12)

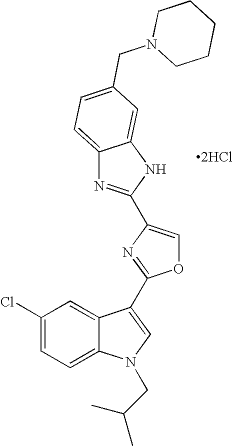

A solution of 2-{2-[5-chloro-1-(2-methylpropyl)-1H-indol-3-yl]-1,3-oxazol-4-yl}-1H-benzimidazole-5-carbaldehyde (0.030 g, 0.07 mmol; may be prepared as described in D17) in dry DCM (1.0 ml) was stirred at room temperature under an atmosphere of argon. Piperidine (0.014 ml, 0.14 mmol) was added to the solution and the solution stirred for 1½ hours at room temperature. After this time, NaBH(OAc)₃ (0.023 g, 0.11 mmol) and AcOH (0.01 ml) were added to the solution. The mixture was stirred at room temperature overnight. After this time, water (few drops) was added to the mixture. Solvent was removed under reduced pressure, and the residue was purified using MDAP. The pure product was treated with 2M HCl/Et₂O.

LCMS Rt=2.39 min, [MH⁺] 488.

### Examples 13-14

The following compounds were prepared using the procedure described in E12:

| Name | Structure | Data |
|---|---|---|
| 2-{2-[5-Chloro-1-(2- methylpropyl)-1H-indol-3-yl]- 1,3-oxazol-4-yl}-5-(1- pyrrolidinylmethyl)-1H- benzimidazole dihydrochloride (E13) | 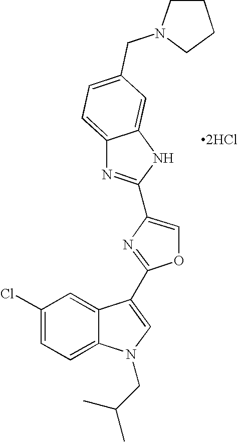 | LCMS Rt = 2.38 min, [MH⁺] 474 |
| [(2-{2-[5-Chloro-1-(2- methylpropyl)-1H-indol-3-yl]- 1,3-oxazol-4-yl}-1H- benzimidazol-5- yl)methyl]ethylamine dihydrochloride (E14) | 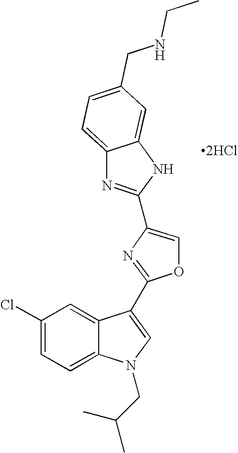 | LCMS Rt = 2.29 min, [MH⁺] 448 |

It is to be understood that the present invention covers all combinations of particular and preferred subgroups described herein above.

### Assays for Determining Biological Activity

The compounds of formula (I) can be tested using the following assays to demonstrate their prostanoid antagonist or agonist activity in vitro and in vivo and their selectivity. Prostaglandin receptors that may be investigated are DP, EP₁, EP₂, EP₃, EP₄, FP, IP and TP.

### Biological Activity at EP₁ and EP₃ Receptors

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable (pClN; BioTechniques 20 (1996): 102-110) vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (pIC₅₀) may then be estimated.

### Binding Assay for the Human Prostanoid EP₁ Receptor

Competition assay using [³H]-PGE2.

Compound potencies are determined using a radioligand binding assay. In this assay compound potencies are determined from their ability to compete with tritiated prostaglandin E₂ ([³H]-PGE₂) for binding to the human EP₁ receptor.

This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5 mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10s burst at full setting), centrifuged at 48,000×g for 20 mins and the pellet containing the membrane fraction is washed (optional) three times by suspension and centrifugation at 48,000×g for 20 mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at −80° C. until required.

For the binding assay the cell membranes, competing compounds and [³H]-PGE₂ (3 nM final assay concentration) are incubated in a final volume of 100 μl for 30 min at 30^{O} C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques to determine the concentration of compound producing 50% inhibition of specific binding (IC₅₀).

### Results

The compounds of examples 1-14 were tested in the binding assay for the human prostanoid EP₁ receptor. The results are expressed as pIC₅₀ values. A pIC50 is the negative logarithm₁₀ of the IC₅₀. The results given are averages of a number of experiments. The compounds of examples 1-5 and 7-14 had a pIC₅₀ value ≧6. More particularly, the compounds of examples 2-3, 5 and 7-13 exhibited a pIC₅₀ value ≧6.5.

The Compounds of Examples 1-14 were tested in the human EP₁ calcium mobilisation assay. The results are expressed as functional pKᵢ values. A functional pKᵢ is the negative logarithms of the antagonist dissociation constant as determined in the human EP₁ calcium mobilisation assay. The results given are averages of a number of experiments. The compounds of Examples 2-11 exhibited a functional pKᵢ value ≧6.0. More particularly, the compounds of Examples 3, 5-9 and 11 exhibited a functional pKᵢ value ≧6.5. The compounds of Examples 1 and 12-14 were inactive in the calcium mobilisation assay.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I): 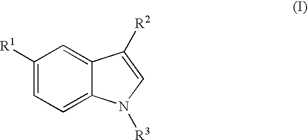
wherein
R¹ represents —CF₃, chlorine or bromine;
R² represents a group of formula (i): 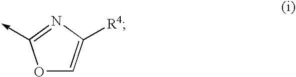
R³ represents isobutyl, —CH₂-cyclopropyl or cyclopentyl;
R⁴ represents —CO—NH—R⁵, —CO-morpholinyl or a group of formula (ii)-(iii): 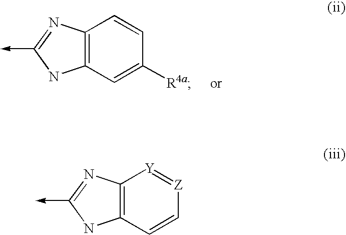
R^{4a} represents hydrogen, —CH₂OH or —CH₂—NR^{a}R^{b};
R⁵ represents hydrogen, pyridyl, morpholinyl, tetrahydropyranyl or —CH₂-tetrahydropyranyl;
R^{a} and R^{b} independently represent hydrogen or C₁₋₃ alkyl or R^{a} and R^{b} together with the nitrogen atom which they are attached form a pyrrolidinyl or piperidinyl ring;
one of Y and Z represents CH and the other represents N;
such that when R² represents a group of formula (I) and R⁴ represents —CO-morpholinyl, R³ represents cyclopentyl;
or derivatives thereof.

2. (canceled)

3. A pharmaceutical composition comprising a compound according to claim 1 or a pharmaceutically acceptable derivative thereof together with a pharmaceutical carrier and/or excipient.

4-5. (canceled)

6. A method of treating a human or animal subject suffering from a condition which is mediated by the action of PGE₂ at EP₁ receptors which comprises administering to said subject an effective amount of a compound according to claim 1 or a pharmaceutically acceptable derivative thereof.

7. A method of treating a human or animal subject suffering from a pain, or an inflammatory, immunological, bone, neurodegenerative or renal disorder, which method comprises administering to said subject an effective amount of a compound according to claim 1 or a pharmaceutically acceptable derivative thereof.

8. A method of treating a human or animal subject suffering from inflammatory pain, neuropathic pain or visceral pain which method comprises administering to said subject an effective amount of a compound according to claim 1 or a pharmaceutically acceptable derivative thereof.

9-11. (canceled)
